(19) United States
(12) Patent Application Publication
(10) Pub. No.: US 2011/0190861 A1
(43) Pub. Date: Aug. 4, 2011

(54) LUMINAL PROSTHESIS

(76) Inventors: Ian Owens Pericevic, Leon (ES); Daniel John Kelly, County Wicklow (IE); Niall Mulvihill, Dublin (IE); Patrick John Prendergast, County Dublin (IE); Saeid Kasiri Ghahi, Dublin (IE)

(21) Appl. No.: 12/734,818

(22) PCT Filed: Nov. 28, 2008
(86) PCT No.: PCT/IE2008/000116

§ 371 Date: Feb. 9, 2011

Related U.S. Application Data
(60) Provisional application No. 60/996,646, filed on Nov. 28, 2007.

(51) Int. Cl. A61F 2/84; A61F 2/82

(52) U.S. Cl. 623/1.11; 623/1.16

(57) ABSTRACT

A luminal prosthesis comprises a plurality of axially arranged radially expandable stent segments 22,23 having coupling parts 20,21 for coupling of the segments 22,23. The segments 22,23 are movable between a collapsed delivery configuration in which the coupling parts 20,21 of the segments are interengaged and a deployed configuration in which the coupling parts 20,21 are disengaged. The stent segments 22,23 have means to delay the disengagement of the coupling parts 20,21 until the stent segments are close to the deployed configuration. A female coupling part 20 comprises an axially extending passageway having an entrance 30 to receive a corresponding axially extending male part 21 of an adjacent stent segment. The delay means may comprise first mating parts 40,41 and second mating parts 50,51 which are axially spaced-apart along the passageway. The second mating parts 50,51 may be located at an end of the passageway remote from the entrance to delay separation. The prosthesis may include link elements 70 to compensate for foreshortening.

### INTRODUCTION

This invention relates to a luminal prosthesis.

While successful in preventing elastic recoil following balloon angioplasty, stenting can result in increased injury and ultimately restenosis in some cases Morton et al, Pathologie Biologie 2004; 52:196-205. Complication rates are higher in long and tortuous vessels, such as the peripheral arteries. In many cases these complications arise due to the inability of a relatively stiff stent to conform to the vessel's curvature.

Peripheral arteries are, generally, highly flexible vessels which undergo various bending, twisting, compression and torsion modes in multiple planes. These modes are particularly pronounced in the superficial femoral artery and popliteal arteries during walking, but may also be observed to a lesser extent in other vessels, for example, in the carotid artery during turning of the head. Therefore, stent flexibility following deployment is a critical design feature for peripheral stents. The relatively large diameters of peripheral vessels requiring stenting will mean a thicker vessel wall causing increased radial compression. Hence, it is important that peripheral stents allow maximum flexibility, whilst providing good support of the vessel wall and resisting radial forces. This has proven difficult to achieve, as it requires a trade-off between stent flexibility and wall support. It has been reported that neither the use of balloon expandable stents, self expanding stents or drug eluting stents have significantly improved patency rates compared to balloon angioplasty alone (Duda et al. J Endovasc Ther 2006; 13: 701-710; Cejna et al, J Vasc Interv Radiol. 2001; 12: 23-31).

Intravascular stents are applied within peripheral or coronary arteries to maintain patency after a balloon angioplasty procedure. During a typical procedure, the stent is expanded from a relatively small diameter to at least that of the vessel wall. Conventional vascular stents often comprise a series of ring-like radially expandable structural members, often referred to as units or segments, which are axially connected by bridge or link elements. The bridge elements function to prevent individual segments from propelling themselves from the delivery system in an uncontrolled fashion as they expand to their full diameter. They also limit stent segments from moving relative to the lumen after expansion, thus preventing segments from overlapping or moving away from one another and creating unsupported gaps. They may also contribute to vessel wall support. The link elements also confer a certain longitudinal rigidity to the stent, thereby potentially contributing to the injury caused by the stent in the vessel wall as it forces the vessel wall to conform to its geometry after expansion.

Vascular injury has consistently been found to determine the degree of restenosis (Schwartz et al., J. Intervent. Card., 7, 355-68, 1994; Hoffman et al., Am. J. Card., 83, 1170-74, 1999). While drug eluting stents have reduced the incidence of restenosis, it is still a clinical problem, and it is recognised that “control of the biological response may also be possible through careful manipulation of the stent design, to enhance the beneficial effect of stent coatings and drugs” (Dean et al., Heart, 91:1603-1604, 2005).

There is therefore a need for an improved stent which will address at least some of these issues.

### STATEMENTS OF INVENTION

According to the invention there is provided a luminal prosthesis comprising a plurality of axially arranged radially expandable stent segments, the segments having coupling parts for coupling of the segments, the segments being movable between:—
a collapsed delivery configuration in which the coupling parts of the segments are interengaged; and
a deployed configuration in which the coupling parts are disengaged,
the stent segments having means to delay the disengagement of the coupling parts until the stent segments are close to the deployed configuration.

In one embodiment the coupling parts comprise a male part and a female part, the male and female parts of adjacent stent segments being interengaged in the collapsed delivery configuration and the male and/or female part comprising the delay means to delay the disengagement of the coupling parts until the stent segments are close to the deployed configuration.

The female part may comprise an axially extending passageway having an entrance to receive a corresponding axially extending male part of an adjacent stent segment, the delay means comprising interengagable mating parts on the male and female parts, the mating parts being spaced axially inwardly of the entrance to the passageway.

In one embodiment there are first mating parts and second mating parts which are axially spaced-apart along the passageway.

The second mating parts may be located at an end of the passageway remote from the entrance.

In one case the second mating parts comprise a head part and a socket part for engagement with the head part. The socket part may comprise a neck which is of reduced dimensions with respect to the head part for retaining the head part in the socket part.

In one embodiment the head part comprises a ball.

The head part may comprise at least one radially extending projection. Preferably the head part comprises a pair of oppositely directed projections.

In one case the projecting portion is of generally rectilinear shape.

In another case the projecting portion is of generally wedge shape.

The projecting portion may be of generally curvilinear shape.

In one embodiment the stent segments are designed so that the male and female parts undergo differential deformation and/or displacement during expansion.

One of the female part or male part may undergo deformation and/or displacement during expansion and the other of the male part or female part does not undergo significant deformation or displacement.

In one case the female part undergoes deformation and/or displacement during expansion and the male part does not undergo significant displacement or deformation.

In another case the male part undergoes deformation and/or displacement during expansion and the female part does not undergo significant displacement or deformation.

In another case both the male and the female parts undergo deformation and/or displacement during expansion.

In one embodiment in the collapsed configuration, the male part extends substantially fully into the female part. In the collapsed configuration, the male part may be configured to substantially fill the female part.

In one case the stent segment comprises a first set of strut elements and a second set of strut elements. The stent segment may comprise a first set of one or more link elements to link at least some of the first set of strut elements to at least some of the second set of strut elements.

The invention also provides an endoprosthesis comprising a plurality of axially arranged radially expandable stent segments, the segments having coupling parts for coupling of the segments, the segments being movable between:—
a collapsed delivery configuration in which the coupling parts of the segments are interengaged; and
a deployed configuration in which the coupling parts are disengaged,
wherein the segment comprises a first set of strut elements, a second set of strut elements, and a first set of one or more link elements to link at least some of the first set of strut elements to at least some of the second set of strut elements and wherein a link element is more flexible than a strut element.

The invention also provides a prosthesis in which a link element extends rather than compresses by virtue of the stent end being engaged with an adjacent stent for a significant part of the expansion process.

In a preferred embodiment the link element is more flexible than the strut element.

The link element may extend in a non-straight manner between the first set of strut elements and the second set of strut elements. The link element may open up or elongate during expansion of the stent.

The link element may extend in a substantially “s”-shape.

The link element may extend in a substantially “w”-shape.

The link element may extend in a substantially “m”-shape.

The link element may extend in a substantially “v”-shape.

In one case a closed cell is defined between the first set of strut elements, the second set of strut elements, and the link elements.

The closed cell may be defined between two strut elements of the first set of strut elements, two strut elements of the second set of strut elements, and two link elements.

The closed cell may be defined between four strut elements of the first set of strut elements, four strut elements of the second set of strut elements, and two link elements.

Preferably the first set of strut elements and the second set of strut elements are connected by at least one link element in the circumferential direction.

In one embodiment at least part of the segment comprises a biodegradable material.

In another embodiment at least part of the segment comprises a radiopaque material.

In a further embodiment there is a coating around at least part of the segment.

The coating may comprise a biologically active agent.

The prosthesis may be expandable by means for example of a balloon inflatable or may be a self-expanding prosthesis.

The prosthesis is particularly suitable for use in a peripheral artery.

In a further aspect the invention provides a method for delivering a luminal prosthesis to a treatment site comprising:—
providing a delivery catheter with a plurality of radially expandable stent segments arranged axially on the delivery catheter, the stent segments having coupling parts which are interengaged;
delivering the catheter to a treatment site;
radially expanding all of the stent segments at the treatment site to a partially expanded configuration in which the coupling parts of the segments remain interengaged; and
further radially expanding all of the stent segments to a deployed configuration in which all of the coupling parts of the stent segments are disengaged.

### BRIEF DESCRIPTION OF THE DRAWINGS

The invention will be more clearly understood from the following description of some embodiments thereof, given by way of example only, with reference to the accompanying drawings, in which:—

FIG. 1 is a view of a stent according to the invention with high conformity coupling in the collapsed configuration;

FIG. 2 is an enlarged view of part of the high conformity coupling system of FIG. 1;

FIG. 3 is a view of a stent with an alternative high conformity coupling mechanism;

FIG. 4 is an enlarged view of portion of the stent of FIG. 3;

FIG. 5 is a cut view of one of the stent segments of FIG. 3;

FIG. 6 is a view of another stent with an alternative high conformity coupling and a W-link;

FIG. 7 is a view of a single-cell stent with an s-link;

FIG. 8 is a view of is a double-cell stent with an s-link;

FIG. 9 is a view of a triple-cell stent with an s-link;

FIG. 10 is a view of portion of a stent with a single-cell/double-cell combination and an s-link;

FIG. 11 is a view of a single-cell stent with a W-link;

FIG. 12 is a view of a double-cell stent with a W-link;

FIG. 13 is a view of a single-cell stent with an modified s-link;

FIG. 14 is a view of a double-cell stent with a V-link;

FIGS. 15 to 17 are enlarged views of a coupling between adjacent stent segments of FIGS. 4 and 5 in a collapsed delivery, a partially expanded, and a deployed configuration respectively;

FIGS. 18 to 21 are enlarged views of a ball and socket coupling between adjacent stent segments in a collapsed configuration, a partially expanded, and deployed configurations;

FIG. 22 is a cut view of a stent segment incorporating the coupling features of FIGS. 18 to 21;

FIG. 23 is a view of a further stent segment incorporating the coupling features of FIGS. 18 to 21;

FIG. 24 is a view of a portion of a stem comprising a number of stent segments;

FIG. 25 is a view of another stent segment incorporating the coupling features of FIGS. 18 to 21;

FIG. 26 is a view of portion of a stent comprising a number of stent segments incorporating the coupling features of FIGS. 18 to 21;

FIGS. 27 to 30 are enlarged views of a coupling between adjacent stent segments with a T shape interlock in a collapsed delivery, partially expanded, and a deployed configuration respectively;

FIG. 31 is a cut view of one stent segment incorporating the coupling features of FIGS. 27 to 30;

FIG. 32 is a view of portion of a stent comprising a number of the stent segments of FIG. 31;

FIG. 33 is a view of portion of a stent comprising a number of stent segments incorporating the coupling features of FIGS. 27 to 30;

FIGS. 34 to 37 are views of further stent segments incorporating the coupling features of FIGS. 27 to 30;

FIG. 38 is a view of a modified stent segment incorporating the coupling features of FIGS. 27 to 30 and some further coupling features;

FIG. 39 is a view of portion of a stent comprising a number of the stent segments of FIG. 38;

FIG. 40 is a view of another stent segment incorporating the coupling features of FIG. 38;

FIGS. 41 to 44 are enlarged views of a coupling between adjacent stent segments in a collapsed delivery, partially expanded, and a deployed configuration;

FIG. 45 is a view of a stent segment incorporating the coupling features of FIGS. 41 to 43;

FIG. 46 is a view of portion of a stent comprising a number of the stent segments of FIG. 45;

FIGS. 47 to 50 are enlarged views of a coupling between adjacent stent segments in a collapsed delivery, partially expanded, and a deployed configuration respectively in which a male part is engaged with a second female part;

FIGS. 51 to 53 are enlarged views of a coupling between adjacent stent segments in a collapsed delivery, partially expanded, and a deployed configuration respectively;

FIGS. 54 to 57 are enlarged views of a coupling between two segments with a trapezoidal interlock in a collapsed delivery, partially expanded, and a deployed configuration respectively;

FIG. 58 is a view of a stent segment incorporating the coupling features of FIGS. 54 to 57; and

FIGS. 59 to 62 are enlarged views of a coupling between adjacent stent segments with a flexible mating system in a collapsed delivery, partially expanded, and a deployed configuration respectively.

### DETAILED DESCRIPTION

Referring to the drawings there are illustrated various luminal prostheses according to the invention. The luminal prosthesis comprise a plurality of axially arranged and radially expandable stent segments 22,23. The segments 22,23 have coupling parts 20,21 for coupling of the segments 22,23. The segments 22,23 are movable between a collapsed delivery configuration in which the coupling parts 20,21 of the segments 22,23 are interengaged and a deployed configuration in which the coupling parts 20,21 are disengaged.

The coupling parts comprise a male part 20 and a female part 21. The male and female parts 20,21 of adjacent stent segments 22,23 are interengaged in the collapsed configuration.

The male and/or female parts comprise a delay means to delay the disengagement of the coupling parts until the stent segments 22,23 are close to the deployed configuration.

The female part 20 comprises an axially extending passageway having an entrance 30 to receive a correspondingly axially extending male part 21 of an adjacent stent segment. The delay means comprises interengagable mating parts on the male and female parts. The mating parts are spaced axially inwardly of the entrance 30 to the passageway.

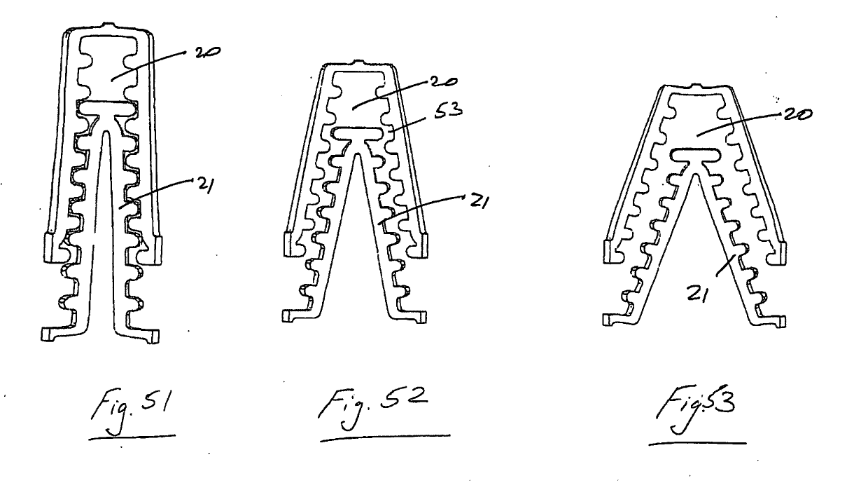

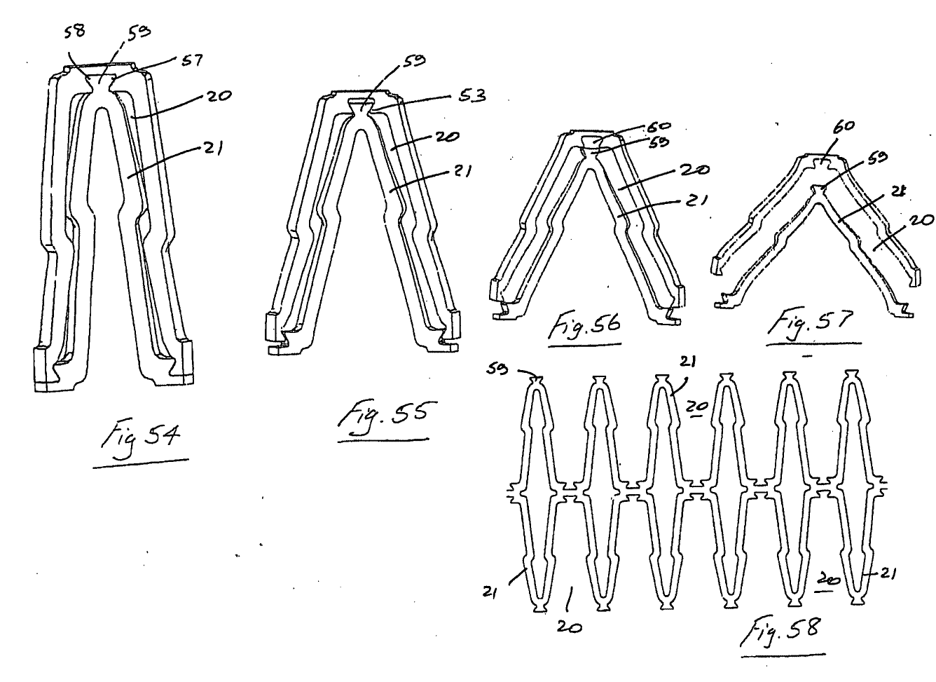

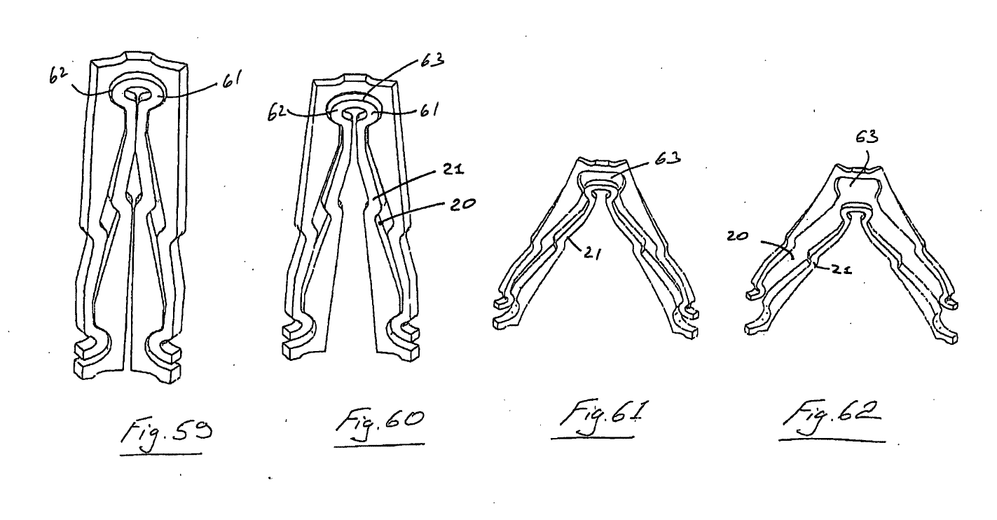

In some embodiments there are first mating parts 40,41 and second mating parts 50,51 which are axially spaced-apart along the passageway. In preferred embodiments the second mating parts 50,51 are located at an end of the passageway 30 remote from the entrance [FIGS. 18 to 62].

The second mating parts may comprise a head part 50 and a corresponding socket part 51 for engagement with the head part 50.

In one case the socket part comprises a neck 53 which is of reduced dimensions with respect to the head part 50. The male part has a neck corresponding to the socket neck part.

The head part may comprise a ball 50 which may be generally spherical in shape [FIGS. 18 to 26].

The head part 50 may comprise at least one radially extending projection. Preferably the head pan comprises two oppositely directed radially extending projections 54,55. In one case the projections 54,55 are of generally rectilinear shape to define a T-section for mating engagement in a correspondingly T-shaped female slot 56 [FIGS. 27 to 53]. In another case the projecting portions are generally wedge shaped 57,58 and the resultant head part 59 is of generally trapezoidal form for mating engagement with a correspondingly shaped female slot 60 [FIGS. 54 to 58]. Alternatively, the projecting parts are generally curvilinear shaped 61,62 for mating engagement with a correspondingly shaped female slot 63 [FIGS. 59 to 62].

In the invention the male and female parts preferably undergo differential deformation and/or displacement during expansion. In some cases one of the parts undergoes significant displacement and/or expansion and the other does not.

The stent of the invention comprises a plurality of “mini-stents”, which are releasably engaged. In other words, when crimped, adjacent mini-stents interlock to form a continuous entity, which will enable them to remain on a balloon and not be prone to sliding once expansion begins. When a certain diameter is reached, the mini-stents will disengage from each other and come in contact with the vessel wall as separate entities acting together. Thus, the series of mini-stents will provide the necessary support to the vessel wall, whilst allowing greater flexibility in the vessel. This increased flexibility should eliminate the high incidence of injury to the vessel wall, as is the case with current solutions, and reduce the resulting levels of restenosis seen today.

The main application envisaged for the stent is for peripheral arteries, where balloon inflatable stents are currently seldom used for larger diameters and lengths; as their increased stiffness causes problems in these highly flexible vessels. Instead, self-expanding stents are commonly utilized. From coronary applications it has been learnt that self-expanding stents provide less support and reliability in situ than classical balloon inflated ones, not to mention less controllability during implantation. Generally, self-expanding stents are a less popular tool amongst interventional cardiologists. To this day, the patency rates of peripheral interventions are significantly lower than those of coronary interventions. This is directly related to that fact that peripheral stents are subjected to significantly different mechanical conditions than coronary stents, making peripheral artery stents significantly more prone to damage and fatigue. Commonly, the physiology of the stenosis is also different. All these factors require superior flexibility and strength for peripheral applications, calling for stents which are specifically designed for peripheral applications, rather than utilizing designs based on coronary solutions. Both failure rates and interventionalist preference are paramount factors.

In addition, the stent of the invention could also be used in cardiovascular applications or for any other bodily lumen, primarily in those cases where stent flexibility is important. These could include, but are not limited to, any artery, vein, esophagus, trachea, colon, biliary ducts or urinary tract.

A balloon inflatable peripheral stenting solution would significantly improve patency rates in peripheral arteries, as well as prove to be a popular tool among vascular interventionalists.

In our invention we use rigid closed cell stent segments that can disengage from each other during expansion as a means of delivering multiple stents from a single balloon. Designing closed cell stent segments as opposed to rings has allowed us to develop such a multiple stenting system. The design and location of the stent mating system ensures that the stents are rigidly interlocked when crimped, but that on expansion the stents can articulate freely relative to each other with zero or minimal contact. No bridge or link elements between adjacent stent segments are necessary to achieve stent deployment.

A stent is a mechanical structure comprising a plurality of tubular, radially expansible rings or sets of strut elements connected to form segments, used for supporting the wall of a blood vessel or another human or animal body lumen. The structure of the stent of the invention can be made up of any combination of straight, curved, arc, s-shaped, z-shaped, v-shaped, u-shaped or loop elements. These elements are connected in such a way as to form a series of segments and eventual connections between these segments on the circumference of the tube they comprise. The manner in which they are connected may form a series of open cell structures, closed cell structures, or a combination of open and closed cell structures.

The basic concept behind the stent of the invention is that it consists of a plurality of composite segments, hereafter referred to as mini-stents, which are not connected by a physical link to adjacent mini-stents. However, the mini-stents are designed in such a way that when crimped they will interlock with adjacent mini-stents, while at higher diameters during the expansion process, the mini-stents will disengage and will not be connected. Hence, the mini-stents will be releasably engaged. This releasable engagement can be achieved without the use of any bridging elements. In the case presented here, the interlocking is achieved when adjacent mini-stents are positioned in-phase. Furthermore, the mating system which allows stents to interlock has been designed in such a way as to minimize stent contact following expansion.

Thus, when crimped, the individual segments will interlock with each other. When deployed within a vessel, this stent will act as a series of separate mini-stents which are physically independent, but continue to function as a single stent in terms of providing the necessary support to the vessel wall, while providing greater wall flexibility.

During balloon expansion, the interlocking of the mini-stents will ensure that a certain distance is maintained between adjacent mini-stents during the deployment process. It will also ensure that the stent acts as a single entity during the initial stages of expansion, providing a means to combat the problem of uncontrolled stent expansion (i.e. axial movement of stents on balloon in case of balloon expandable stents).

The deployment of this stent will be achieved primarily by balloon expansion, but may also be achieved by self-expansion; i.e. through the use of shape-memory materials. The deployment of the stent can be achieved from a single device. In the case of self-expansion, the mini-stents will be deployed by the use of a delivery device.

The concept of the stent and variations in the design are illustrated in the drawings. Particular attention is drawn to the mating of the stent segments.

A stent of the invention comprises a plurality of releasably engageable segments. The coupling mechanism may allow some travelling space. Further modifications are possible in cases where coupling is only necessary on one side of the mini-stent. Some images represents the “cut” shape of the stent coupling, meaning the stents will further interlock following stent crimping.

The stent is movable between a collapsed configuration and an expanded configuration. Some images illustrate various stents according to the invention in a cut form, that is not fully collapsed. Other drawings illustrate various stents according to the invention in the fully collapsed form. The stent may be balloon inflatable or self-expanding.

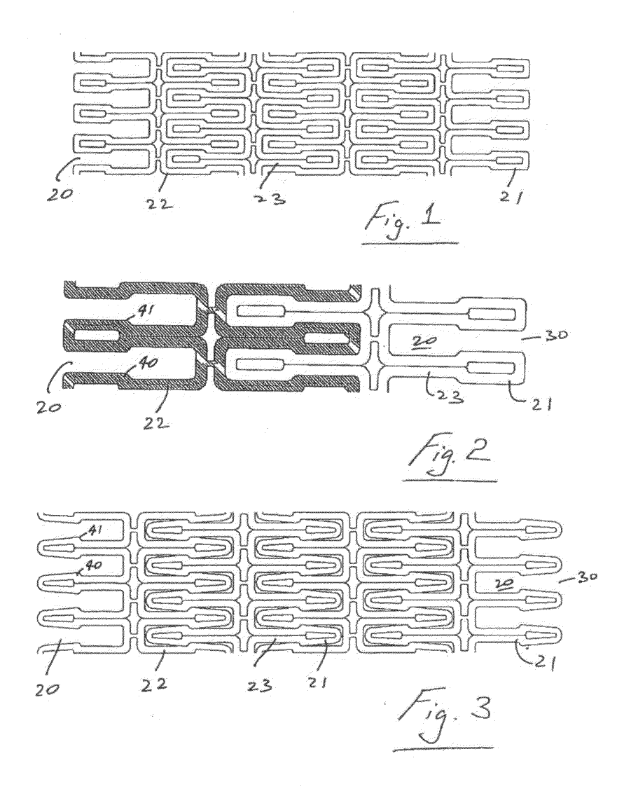

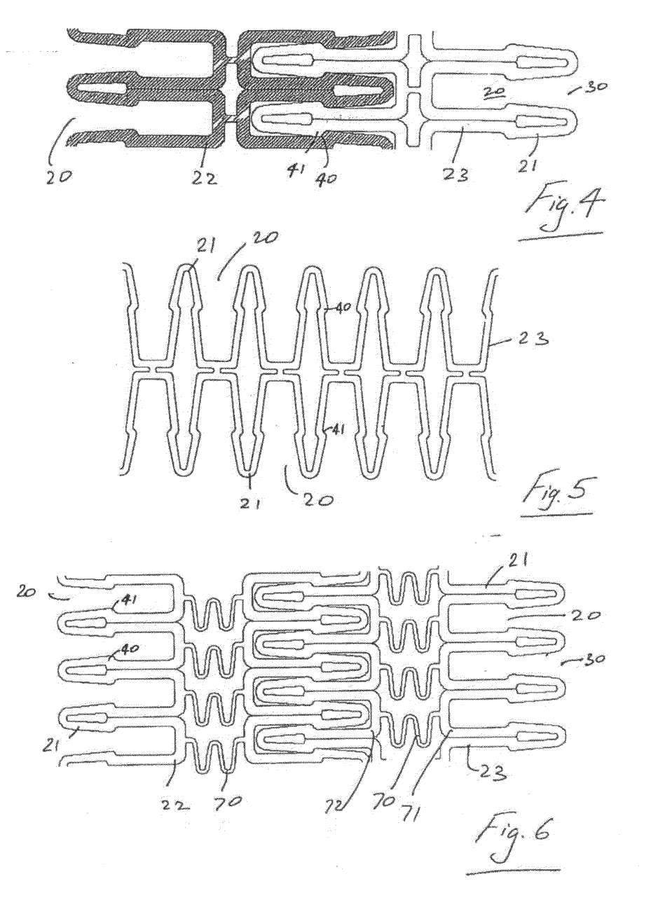

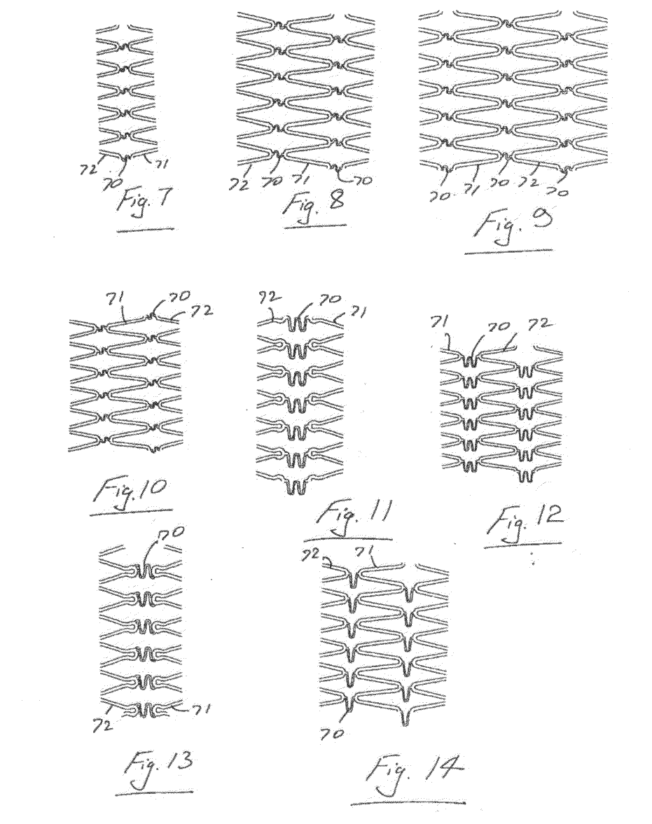

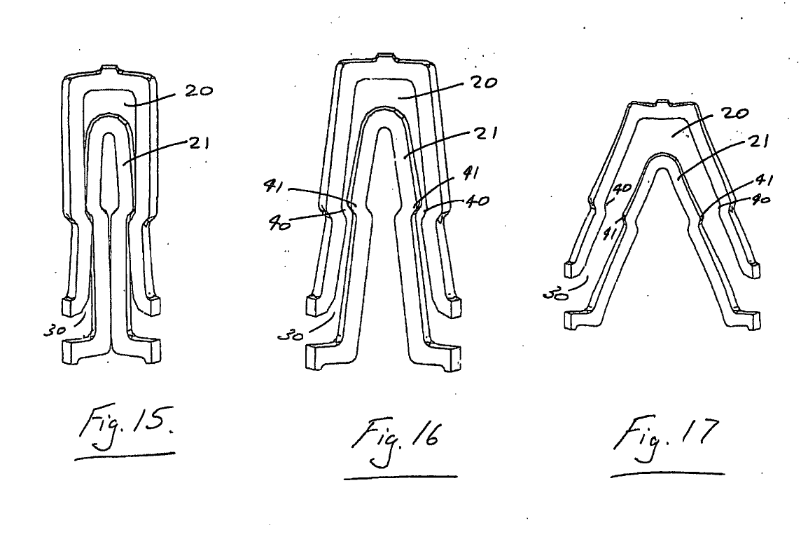

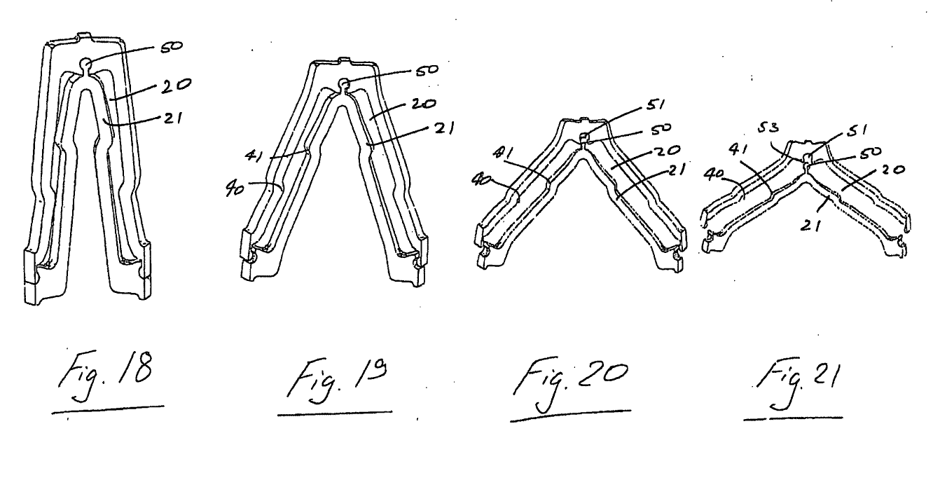

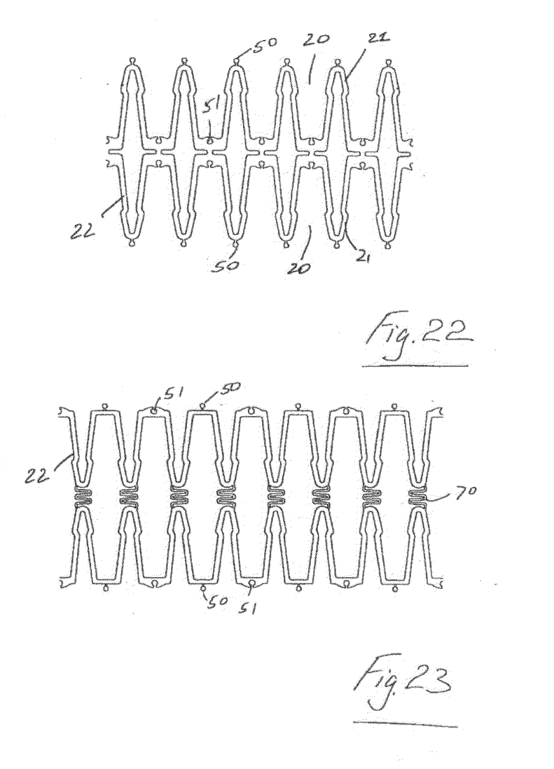

Referring to FIGS. 1 and 2 there is illustrated a stent with a high conformity coupling. The stent cells are designed in such a way as to facilitate alternating male 21 and female 20 sections for mating. The male section 21 is embedded deep into the female section 20. During expansion, movement of individual cells is restricted, also minimizing overall stent foreshortening as a result. It will be appreciated that this design can be modified to include one or more cells.

In this case, in the collapsed configuration the male part 21 extends fully into the female part 20 to fill the female part 20.

FIGS. 3 to 5 illustrate a stent with an alternative high conformity coupling. In this case the male ends 21 are tapered and slightly shortened to facilitate mating. This modification may be achieved in a number of ways with various curvatures and the like on the male side 21. In this case the leading end of the male part 21 is tapered inwardly.

The stent may have a single-cell design consisting of a single-cell stent segment with adjacent interlocking segments. Each segment may comprise a first set of strut elements; a second set of strut elements, and a set of link elements to link the first set of strut elements to the second set of strut elements. This arrangement results in a closed cell being defined between two strut elements of the first set of strut elements, two strut elements of the second set of strut elements, and two link elements.

To compensate for foreshortening of the stent segments 22,23 on movement between the collapsed delivery configuration and the expanded configuration the stent preferably incorporates link elements 70 which extend in a non-straight manner between strut elements of the stent segments 22,23. The link elements 70 may be of any desired shape and configuration. In FIG. 6 the link elements 70 are generally of w-shape. The stem shapes shown in FIGS. 6 to 14 are for illustrative purposes. They may be of any suitable shape and configuration such as those illustrated in any of FIGS. 1 to 5 and FIGS. 15 to 62. FIG. 7 illustrates a single-cell stent with an s-link 70. This provides improved trackability whilst the stent is crimped on a balloon. In this case the link element 70 is more flexible than the strut elements 71. As illustrated in FIG. 7 the link element 70 extends between the first set of strut elements 71 and the second set of strut elements 72 in a non-straight manner, in this case in a “s”-shape. The extension of the link element during expansion is facilitated by the delayed separation of the stents provided by the coupling elements.

FIG. 8 shows a double-cell stent with an s-link 70. FIG. 9 illustrates a triple-cell stent with an s-link 70. FIG. 10 shows another stent which has a single-cell/double-cell combination with an s-link 70. The mini-stents can be used in various combinations such as those illustrated in this figure, where longer mini-stents are placed at each end of the entire stent to provide extra stiffness during deployment.

FIG. 11 illustrates a single-cell stent with a W-link 70. This provides a further variation of a linking strut. In this case the link element 70 extends between the first set of strut elements 71 and the second set of strut elements 72 in a “w”-shape. FIG. 12 shows a double-cell stent with a W-link 70.

FIG. 13 illustrates a single-cell stent with a modified s-link 70. This is a further variation of a linking strut. In this case the link element 70 extends between the first set of strut elements 71 and the second set of strut elements 72 in an “m”-shape.

FIG. 14 shows a double-cell stent with a V-link 70. This is a still further variation of a linking strut. Moving the locations of the links facilitates control over the time of mini-stent disengagement during expansion. In this case the link element 70 extends between the first set of strut elements 71 and the second set of strut elements 72 in a “v”-shape.

The invention provides interlocking stents that only separate once a certain minimum diameter is reached during the expansion process. The design and location of the stent mating system ensures that the stents are rigidly interlocked when crimped, but that on expansion the stents can articulate freely relative to each other with zero or minimal contact. Early separation of segments may cause rotation of segment during expansion as well as longitudinally displacement. Early separation may also prevent the link elements from straightening out during expansion to minimise foreshortening.

Each lock contains a female part 20 and a male part 21. The male part 21 is surrounded by the female part 20. Separation of such initially interlocking stents during expansion only occurs when the female part 20 expands to provide an opening large enough to release the male part, or if the male component contracts during expansion such that it can pass through the female part 20. The point at which separation of interlocking stents occurs during expansion can be controlled by controlling the relative displacement and/or deformation of the female and male components 20,21 during expansion.

In the stent of FIGS. 1 to 6 and 15 to 17 control of stent separation is achieved by altering the ratio between the height of male part 21 and the female part 20 and the ratio between the width of the male and female components 20,21 of the mating system. By increasing the ratio of it is possible to delay separation. However, there are physical limits to how much these can be increased by. For example, if the height of the male component 21 is too great, it will not be able to elongate normally during crimping of the stent, as it will be constrained by the female component. In addition, if the male and female components 20,21 overly conform in the crimped configuration, there may be a tendency for the male part 21 to push the female part 20 longitudinally as it opens up during expansion, which could contribute a reduction in control during stent delivery.

In the stent of FIGS. 18 to 26 the male part 50 is designed to undergo minimal deformation during expansion. Therefore separation will occur once the opening in the female part 51 displaces or deforms to a length greater than the diameter of the head of the male component 50. The stent has been designed to ensure that this only occurs once a significant radial expansion of the stent has occurred. This is achieved by locating the female part 51 in a region of the stent that experiences relatively small displacement/deformations during expansion. The stent system separates much later during the expansion process than the stent of FIGS. 1 to 17, therefore the risk of individual stents moving longitudinally along the balloon or rotation of the stents during expansion (i.e. uncontrolled delivery) is limited. Given that the male component 50 does not displace or deform significantly during expansion, the risk that one segment is pushed away by another segment (shooting) is minimised.

FIGS. 18 to 21 show the engagement of two stent segments. There are two types of coupling involved. The coupling of FIGS. 15 to 17 is modified to provide less pushing of the segments against each other during expansion. A second coupling system is a fixed male 50, flexible female 51 mating placed at the low deformation end of the female part 20.

FIG. 19 illustrates that after initial expansion the coupling system 40,41 is disengaged but the secondary mating system 50,51 are still coupled.

The secondary mating system 50,51 only become disengaged at the larger diameter close to full deployment as illustrated in FIG. 20. The amount of delay can be controlled by altering the ratio between the diameter and the neck width of the male part 50 with corresponding changes to the female part 51.

FIG. 21 illustrates the final deployed configuration. The mating systems are disengaged. The movement of stent is controlled by balloon after disengagement.

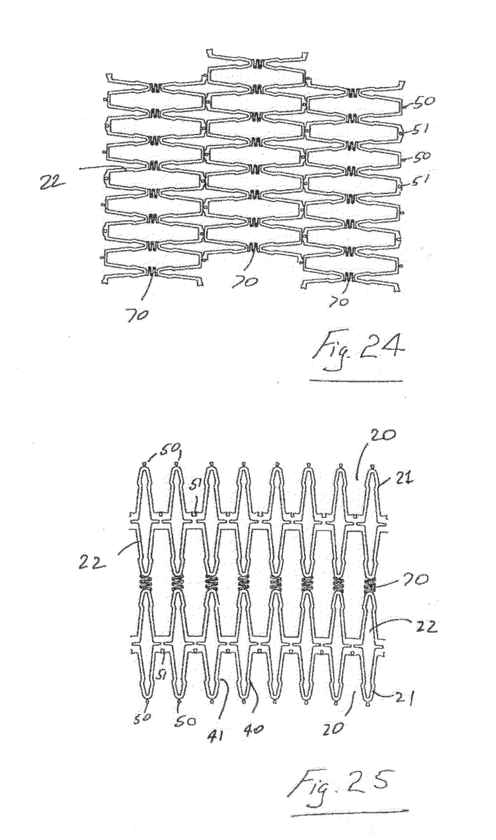

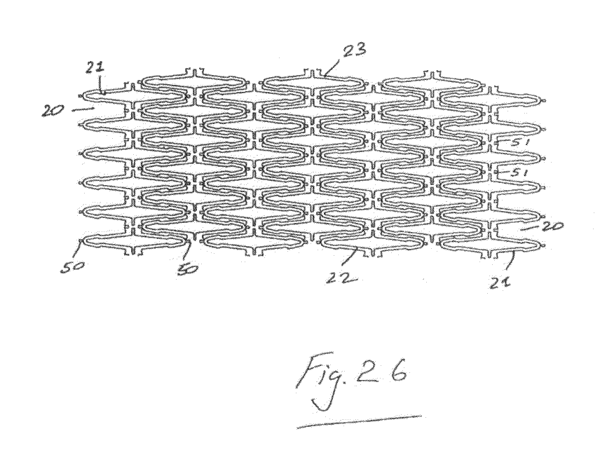

FIG. 22, illustrates a full segment containing the mating system described above. The secondary mating system 50,51 is located such that the female part undergoes relatively small displacements compared with other regions of the stent in order to delay separation with the male part. Delayed separation ensures that the individual stents act as a single unit during expansion, preventing uncontrolled movement of one or more stent segments during delivery. The edges of the female part 51 are close to the neck of male part 50 in order to provide enhanced control. The female part 51 is still part of the stent therefore it opens with deformation of the stent rather than being forced open by the male part 50. In the absence of this feature, disengagement could occur due to displacement of male part longitudinally along the delivery device with respect to the female part. In such a situation, the force applied to the female part by the male part could potentially force the female part open, resulting in uncontrolled movement and/or separation of adjacent stents. As already described, in the presented stent mating system the female part expands due to radial deformation of the stent. In the present interlocking system, the separation is controlled by the extent of radial expansion of the stent. This is a particularly important and advantageous aspect.

FIG. 23 illustrates a segment of a mini-stent having a mating system for segments with no overlap. The mating system is the same as that of FIGS. 18 to 22. There are link elements 70 as described above. Engagement of three segments is shown in FIG. 24.

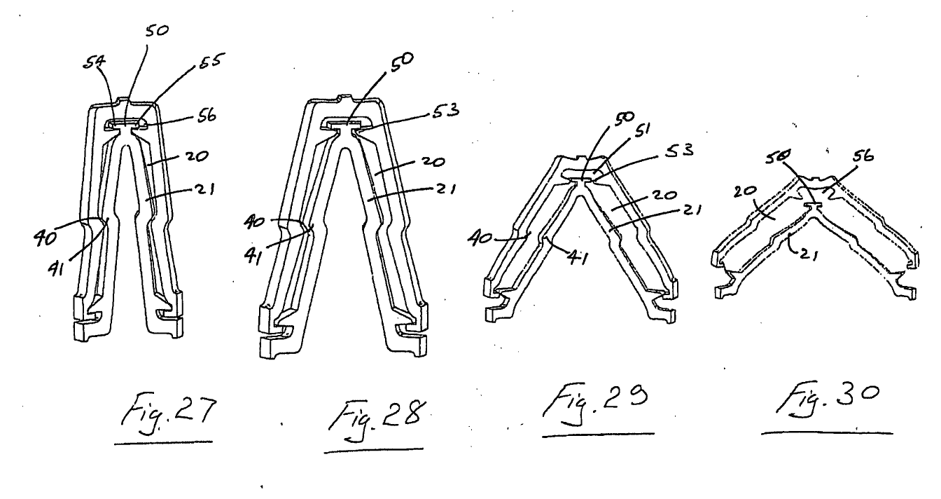

FIG. 25 is a double cell size with the same mating system as FIG. 22. The double cell size helps to avoid flipping of mini stents under different loading once it is placed in an artery. The link 70 between them can be different shapes. The interlink 70 associated with the interlocking system reduces the foreshortening of the segment. The delayed separation of interlocking stents provided by the mating systems ensures that the interlink straightens rather than compresses by preventing uncontrolled axial movements of stents along the delivery device.

The separation can be delayed even further by modifying the shape of the male part 50. Referring to FIGS. 27 to 37 a ‘T’ shaped male component 50 ensures delayed separation if it undergoes relatively small displacements and deformations during expansion. The delay can be controlled by the ratio between the length of the top of the ‘T’ and the neck thickness of the “T”. Greater delay can be achieved using a ‘T’ interlock compared to a circular interlock but the male component may undergo greater deformation before separation. This could possibly lead to early separation if the top of the ‘T’ is forced to take a ‘V’ or ‘U’ shape during expansion.

FIGS. 27 to 30 show the engagement of two cells of two segments having two different coupling systems. The second coupling system 50,56 is a T shape coupling system. As with the circular mating system of FIGS. 18 to 26 more delay is provided. The T shape lock can give more delay control than a circular shape. The whole segment is shown in FIG. 31. It will be noted that the introduced mating system retains the symmetry of the segment. It is important to have the symmetry of mini-stents in order to provide a uniform deformation after expansion and recoiling.

FIG. 27 shows engagement of two cells of two segments at partial expansion when both coupling systems are still engaged.

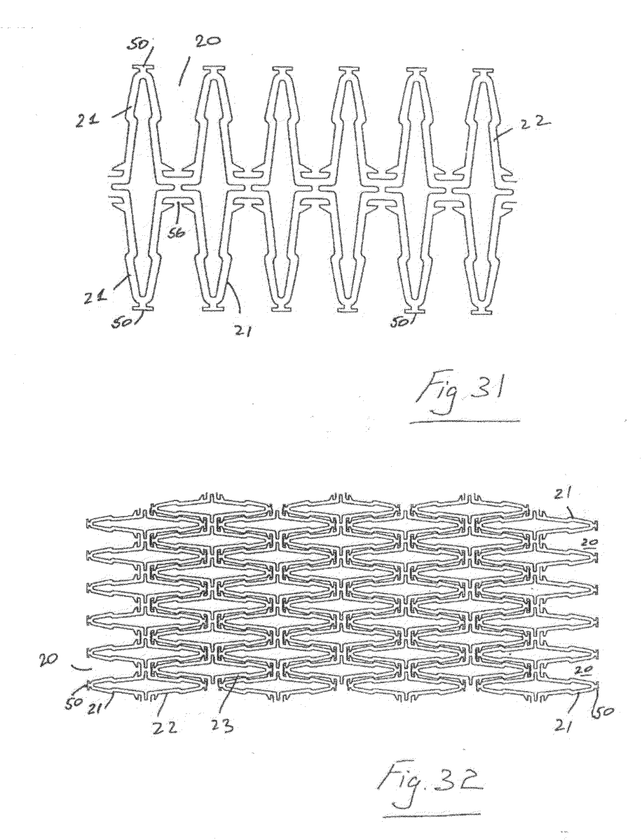

FIG. 28 demonstrates the disengagement of the first coupling system 40,41. In this case the male part 21 opens as well as female part 20. At disengagement, the outer width of the male part 21 is smaller than the inner width of the female part 20.

FIG. 29 shows the disengagement of the secondary coupling system 50,51. It occurs after the disengagement of the first coupling system 40,41. At the separation time the female part opens to a size equal or greater than the width of the head part of the T 50.

FIG. 30 shows a partial expansion after total disengagement.

FIG. 31 illustrates a segment containing a T shape mating system.

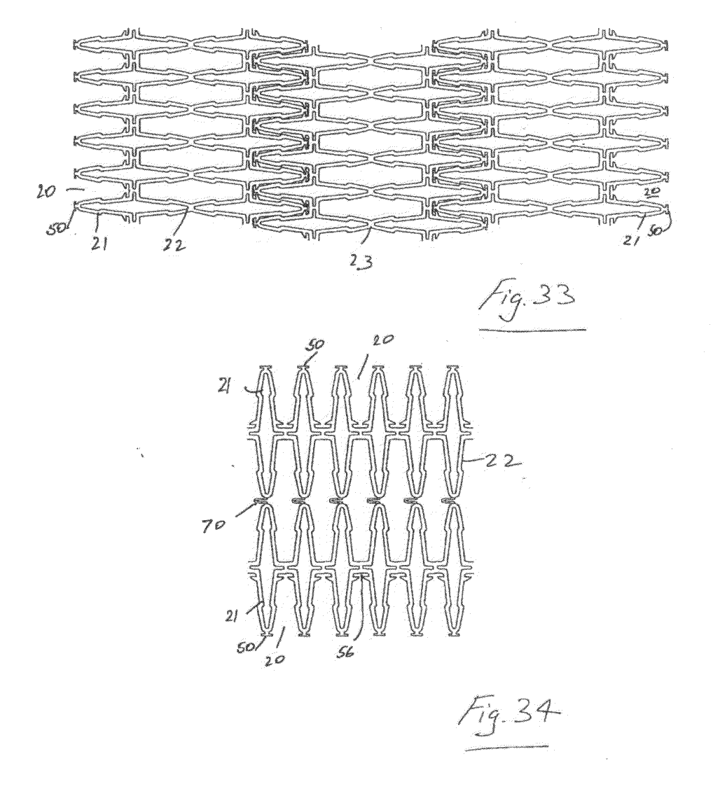

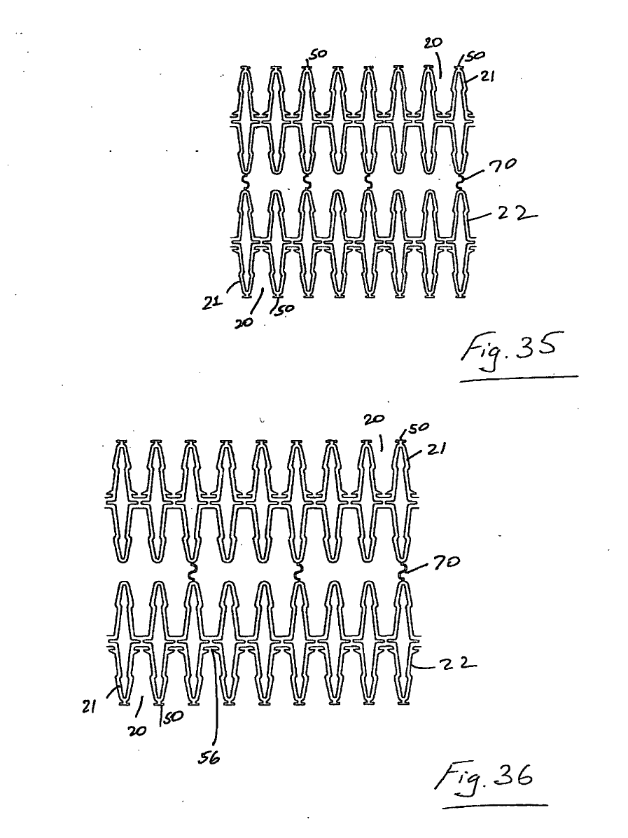

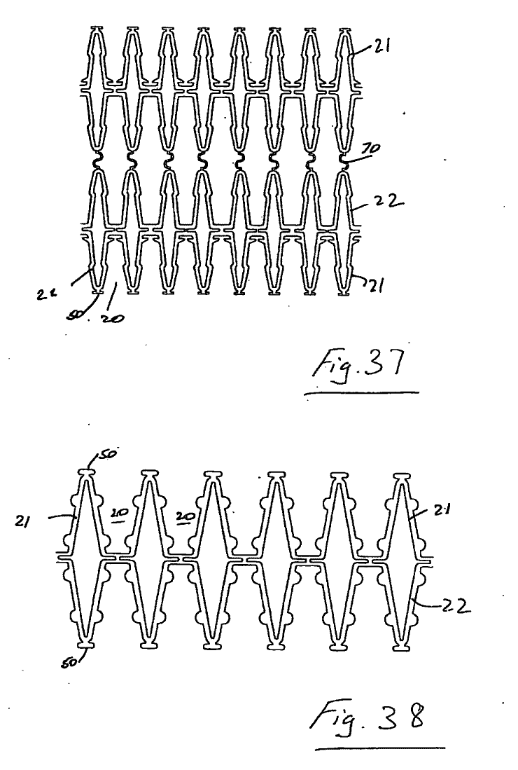

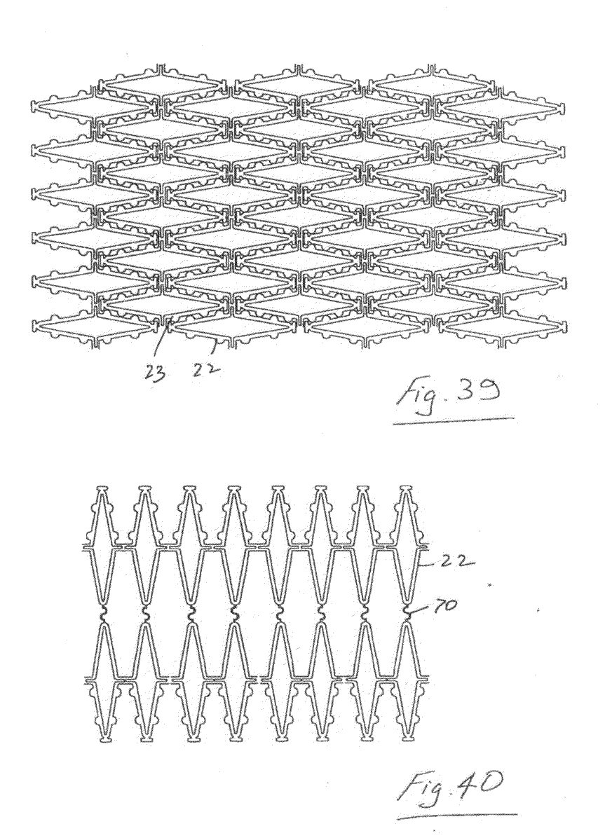

Engagement of seven segments is shown in FIG. 32.

FIGS. 33 to 37 illustrate the mating system in a double sized segment with different types of interlinks. The interlinks between the double cell segments can provide less foreshortening as well as the longer length of the stent cause less risk of flipping of the stent.

FIG. 33 is a double cell segment with no interlink. How ever a longer length after deployment can be provided compared to the single cell segment.

FIG. 34 is a double cell segment using a U interlink 70. The interlink reduces foreshortening.

FIGS. 35 and 36 is double cell segment with partial interlinking. This is more flexible compared to the stent of FIG. 34.

FIG. 37 is double size cell with S interlinks 70. The S interlink may give enhanced control over foreshortening than a U interlink.

FIGS. 38 to 62 illustrate multiple mating systems in one stent. In this system depending on the delay required the male part can be engaged with different female part. In order to get the longest delay in separation, the male part may be engaged with the last female part nearest the end of the passageway 30. The first female component provides the earliest separation due to the large deformation. The desired delay in separation can thereby be selected.

Referring to FIGS. 38 to 40 there is illustrated a stent segment with multiple female components 80 located along the length of the stent. In this way, separation can be controlled by crimping the male part of the stent into different female parts. By crimping the male component (at the apex of the stent) into the female component formed at the base of the stent, stent separation is delayed.

FIG. 38 shows the mating system with two female components and one male component. The male component can be engaged with each female component however the inner female part provides more delay and the outermost one may be used if earlier separation is required.

FIG. 39 shows engagement of segments of FIG. 38 at cut configuration. The male part is engaged with the inner female to provide the most delay in separation.

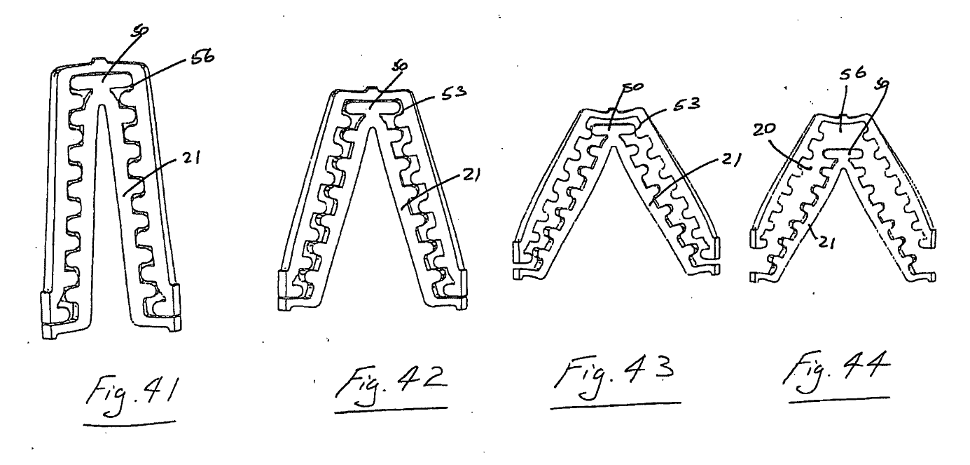

Referring to FIGS. 41 to 44 earlier separation can be achieved by crimping the same male component into one of the female parts 80 formed when two adjacent hemi-spheres come into contact during crimping. This also illustrates the importance of designing the stent to ensure that the female part 80 is in a region of low displacement if separation of multiple stents is to be delayed.

FIG. 40 is a double cell segment including the step control of FIG. 38. The interlink 70 between the segments reduces foreshortening.

FIG. 41 shows the engagement of two multiple female segments at crimped configuration.

FIG. 42 is a partial expansion of the segments of FIG. 41.

FIG. 43 shows the separation of male and female component.

FIG. 44 is the partial expansion of the segments of FIG. 43 after disengagement.

FIG. 45 is a segment of stent at cut configuration containing multiple female part.

FIG. 46 is an example of engagement of the segments having multiple female components at cut configuration.

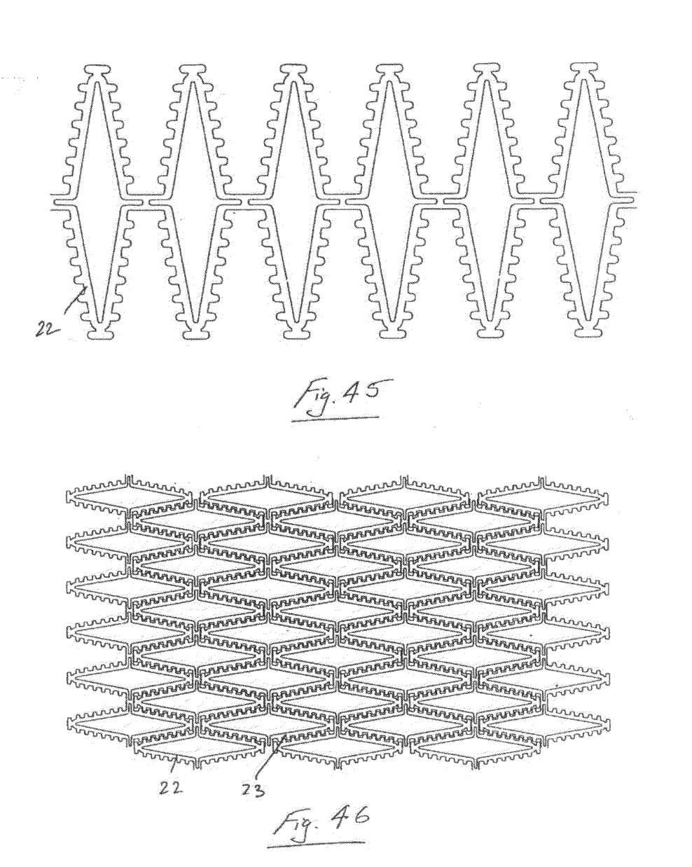

FIG. 47 shows a close view of the crimped segments of stent having multiple female components when the male part is engaged with the second female part. The disengagement occurs earlier than the stent of FIG. 41.

FIG. 48 shows the partial expansion of the stent before disengagement.

FIG. 49 shows the partial expansion at the disengagement of the mating system.

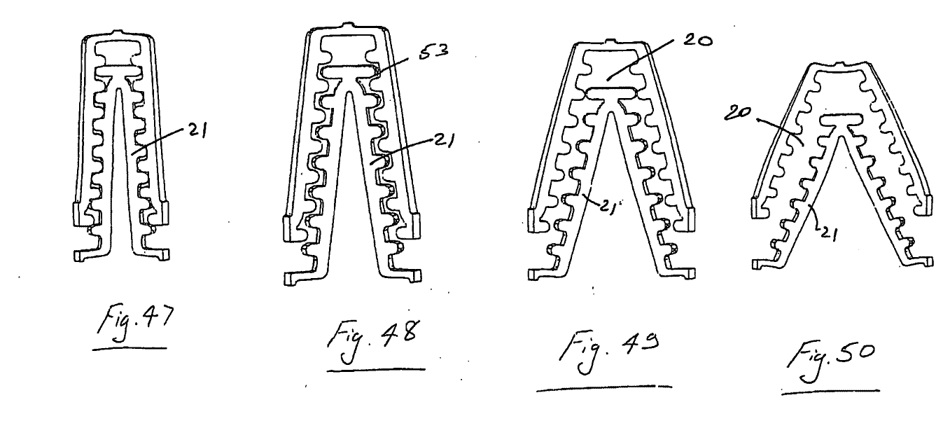

FIG. 50 is the expanded configuration after disengagement.

FIGS. 51 to 53 show the crimped and partial expansion of stents having multiple female components. The male part is engaged with the third female part for earlier separation as described above.

Referring to FIGS. 54 to 58 another variation is a trapezial shaped male component. The male part of this stent design will undergo lower deformation compared to the male part of the ‘T’ design during expansion.

FIG. 54 illustrates a partial expansion of two segments of the stent. The stent contains two different coupling system. A first flexible coupling system stent and a second mating system which is the fix-male flexible female stent.

FIG. 55 shows the separation of the first coupling system.

FIG. 56 demonstrates the disengagement of the secondary mating system at an enlarged diameter of the stent. The separation happens when the female part expands more than the larger base of the trapezium. This secondary mating system controls the separation. At this stage the balloon is inflated sufficiently to prevent rotation and axial movement of stent.

FIG. 57 is a partially expanded stent after disengagement of the secondary mating system.

FIG. 58 shows a segment having a trapezoidal interlocking system. Each segment includes males as well as females.

With the design variations of FIGS. 54 to 62 the point during expansion when separation occurs can be further controlled by increasing or decreasing the width of the head of the male part relative to the neck of the male part and the opening of the female part.

As illustrated for example in FIGS. 59 to 62 the male component can also be designed to expand during expansion, therefore separation is delayed as the amount of expansion the female component must experience prior to separation must also increase.

FIG. 59 shows the engagement of two segments of stents having original interlock and additional flexible interlocking system at crimping configuration.

FIG. 60 shows the disengagement of the first interlocking system while the secondary mating system is still engaged.

FIG. 61 is the segments of the stents when the secondary mating system is disengaged.

FIG. 62 is a partial expansion of the stent after disengagement of the segments. More delay can be achieved compared to the same design as fixed male/flexible female.

Any of the above designs can be altered by making a double cell segments or making longer cells to increase the total length of the stent. This may be necessary to prevent stents ‘flipping’ following delivery into an artery. An optimum range of segment length can prevent the stent flipping as well as keep the flexibility of whole stent.

In the invention decoupling of two stent segments does not occur until significant radial expansion of the stent segments is achieved. The male and female parts of the coupling system are highly conforming. Therefore axial connection in both directions (e.g. up or down the balloon or delivery system) is maintained between adjacent stents until radial expansion of stent occurs, and the opening provided by female part is greater that the outer diameter of the male part. As expansion of the stent segments occurs, significant foreshortening will occur (e.g. FIG. 15-17). Therefore to prevent such foreshortening when delivering multiple stents, link elements that undergo elongation during expansion should preferably be incorporated into the stent design.

In the invention, to further delay separation of adjacent stent segments, the mating system is located in a region that undergoes relatively small displacements during expansion, thereby ensuring axial connection between stent segments until late in the expansion process (see FIG. 18-21 and FIG. 27-30). In the invention the mating system is located in region of low displacement, which is achieved by having substantial interleaving of adjacent stents. The mating system is located in the body of the stent, and not at the end. To achieve this, the stents substantially interleave/penetrate each other in the collapsed configuration. The advantage of this is that the stents will still interleave once expanded, thereby providing greater vessel wall support. In the invention adjacent stents interleave substantially for delayed uncoupling of mating system to occur. In addition, the conforming nature of the mating system used in the invention substantially prevents relative movement of adjacent stents in either the positive or negative axial direction.

In the invention there may be two couplings between adjacent stents, one formed by stent bodies when they are collapsed around each other, and a second at the ends of the stents. The stent bodies are designed to be reasonably conforming, such that they guide the male part of the mating system at the end of each stent body into the corresponding female component on the adjacent stent during crimping/collapsing of stent.

The invention may be used in conjunction with a biologically active agent to inhibit hyperplasia along with coatings and compounds to control their release. The biologically active agents may include, but are not limited to, antineoplastic drugs, antibiotics, immunosuppressants, nitric oxide sources, estrogen and estradiols.

The stent may be manufactured from a number of metallic and polymeric materials, as well as biodegradable compounds and other materials which degrade over time once deployed within the lumen.

The design of the stent may be altered to create radiopaque markers at distinct locations; i.e. the elements may be altered or additional ones added to include materials such as gold or any other radiopaque material.

In order to better control mechanical behaviour of the stent, the relative dimensions of the individual elements may vary within a single mini-stent, or between corresponding elements on the different mini-stents. For example, some elements of the outer mini-stents of the stent may be thicker than corresponding elements on mini-stents at the centre of the stent.

The invention is not limited to the embodiments hereinbefore described, with reference to the accompanying drawings, which may be varied in construction and detail.

1-53. (canceled)

54. A luminal prosthesis comprising a plurality of axially arranged radially expandable stent segments, the segments having coupling parts for coupling of the segments, the segments being movable between:—
a collapsed delivery configuration in which the coupling parts of the segments are interengaged; and
a deployed configuration in which the coupling parts are disengaged,
the stent segments having means to delay the disengagement of the coupling parts until the stent segments are close to the deployed configuration.

55. The prosthesis as claimed in claim 54 wherein the coupling parts comprise a male part and a female part, the male and female parts of adjacent stent segments being interengaged in the collapsed delivery configuration and the male and/or female part comprising the delay means to delay the disengagement of the coupling parts until the stent segments are close to the deployed configuration.

56. The prosthesis as claimed in claim 55 wherein the female part comprises an axially extending passageway having an entrance to receive a corresponding axially extending male part of an adjacent stent segment, the delay means comprising interengagable mating parts on the male and female parts, the mating parts being spaced axially inwardly of the entrance to the passageway.

57. The prosthesis as claimed in claim 56 comprising first mating parts and second mating parts which are axially spaced-apart along the passageway.

58. The prosthesis as claimed in claim 57 wherein the second mating parts are located at end of the passageway remote from the entrance.

59. The prosthesis as claimed in claim 57 wherein the second mating parts comprise a head part and a socket part for engagement with the head part.

60. The prosthesis as claimed in claim 59 wherein the socket part comprises a neck which is of reduced dimensions with respect to the head part for retaining the head part in the socket part.

61. The prosthesis as claimed in claim 60 wherein the head part comprises a ball.

62. The prosthesis as claimed in claim 60 wherein the head part comprises at least one radially extending projection.

63. The prosthesis as claimed in claim 62 wherein the head part comprises a pair of oppositely directed projections.

64. The prosthesis as claimed in claim 62 wherein the projecting portion is of generally rectilinear shape.

65. The prosthesis as claimed in claim 62 wherein the projecting portion is of generally wedge shape.

66. The prosthesis as claimed in claim 62 wherein the projecting portion is of generally curvilinear shape.

67. The prosthesis as claimed in claim 55 wherein the male and female parts undergo differential deformation and/or displacement during expansion.

68. The prosthesis as claimed in claim 67 wherein one of the female part or male part undergoes deformation and/or displacement during expansion and the other of the male part or female part does not undergo significant deformation or displacement.

69. The prosthesis as claimed in claim 68 wherein the female part undergoes deformation and/or displacement during expansion and the male part does not undergo significant displacement and/or deformation.

70. The prosthesis as claimed in claim 68 wherein both the male and the female parts undergoes deformation and/or displacement during expansion.

71. The prosthesis as claimed in claim 68 wherein the male part undergoes deformation and/or displacement during expansion and the female part does not undergo significant displacement and/or deformation.

72. The prosthesis as claimed in claim 55 wherein in the collapsed configuration, the male part extends substantially fully into the female part.

73. The prosthesis as claimed in claim 72 wherein in the collapsed configuration, the male part is configured to substantially fill the female part.

74. The prosthesis as claimed in claim 54 wherein the stent segment comprises a first set of strut elements and a second set of strut elements.

75. The prosthesis as claimed in claim 74 wherein the stent segment comprises a first set of one or more link elements to link at least some of the first set of strut elements to at least some of the second set of strut elements.

76. The prosthesis as claimed in claim 75 wherein the link element is more flexible than the strut element.

77. The prosthesis as claimed in claim 75 wherein the link element extends in a non-straight manner between the first set of strut elements and the second set of strut elements.

78. The prosthesis as claimed in claim 75 wherein a closed cell is defined between the first set of strut elements, the second set of strut elements and the link elements.

79. The prosthesis as claimed in claim 74 wherein the first set of strut elements and the second set of strut elements are connected by at least one link element in the circumferential direction.

80. A luminal prosthesis comprising a plurality of axially arranged radially expandable stent segments, the segments having coupling parts for coupling of the segments, the segments being movable between:—
a collapsed delivery configuration in which the coupling parts of the segments are interengaged; and
a deployed configuration in which the coupling parts are disengaged,
the segments having coupling parts for coupling of the segments, the coupling parts comprising a male part and a female part, the male and female parts of adjacent stent segments being interengaged in the collapsed delivery configuration,
the female part comprising an axially extending passageway having an entrance to receive a corresponding axially extending male part of an adjacent stent segment,
the male and female parts having first mating parts and second mating parts which are spaced axially inwardly of the entrance to the passageway in the delivery configuration and which are axially spaced-apart along the passageway
the first and second mating parts delaying the disengagement of the coupling parts until the stent segments are close to the deployed configuration.

81. An endoprosthesis comprising a plurality of axially arranged radially expandable stent segments, the segments having coupling parts for coupling of the segments, the segments being movable between:—
a collapsed delivery configuration in which the coupling parts of the segments are interengaged; and
a deployed configuration in which the coupling parts are disengaged,
wherein the segment comprises a first set of strut elements, a second set of strut elements, and a first set of one or more link elements to link at least some of the first set of strut elements to at least some of the second set of strut elements and wherein a link element is more flexible than a strut element.

82. A method for delivering a luminal prosthesis to a treatment site comprising:—
providing a delivery catheter with a plurality of radially expandable stent segments arranged axially on the delivery catheter, the stent segments having coupling parts which are interengaged;
delivering the catheter to a treatment site;
radially expanding all of the stent segments at the treatment site to a partially expanded configuration in which the coupling parts of the segments remain interengaged; and
further radially expanding all of the stent segments to a deployed configuration in which all of the coupling parts of the stent segments are disengaged.
